(19) United States
(12) Patent Application Publication
(10) Pub. No.: US 2016/0100954 A1
(43) Pub. Date: Apr. 14, 2016

(54) FUSION DEVICE AND ASSOCIATED METHODS

(71) Applicant: SpineSmith Partners, L.P., Austin, TX (US)

(72) Inventors: Mustasim Rumi, Round Rock, TX (US); Richard J. Kana, Lexington, TX (US)

(21) Appl. No.: 14/975,324

(22) Filed: Dec. 18, 2015

Related U.S. Application Data
(63) Continuation-in-part of application No. 13/135,675, filed on Jul. 12, 2011, now Pat. No. 9,241,810.
(60) Provisional application No. 61/363,641, filed on Jul. 12, 2010.

(51) Int. Cl. A61F 2/44; A61F 2/46

(52) U.S. Cl. CPC A61F 2/447; A61F 2/4611; A61F 2002/4475; A61F 2002/448; A61F 2002/30383

(57) ABSTRACT

An interbody fusion device for fusion of vertebrae, and methods thereof. The device includes a first piece having upper and lower surfaces configured to engage the endplates of two opposing vertebrae, and a second piece that is adapted to either be inserted into the first piece or to be used independently of the first piece. The interbody fusion device can include a surface treatment, such as a porous surface treatment, to promote fusion between the device and a vertebra. The interbody fusion device may also include one or more depressions. The one or more depressions may be used in combination with a material to promote fusion between the device and a vertebra.

### CROSS REFERENCES TO RELATED APPLICATIONS

This application is a continuation-in-part of U.S. patent application Ser. No. 13/135,675 filed on Jul. 12, 2011, which claims priority to U.S. Provisional Patent Application No. 61/363,641 filed Jul. 12, 2010. U.S. patent application Ser. No. 13/135,675 and U.S. Provisional Patent Application No. 61/363,641 are incorporated herein by reference in their entirety as if fully set forth herein.

### BACKGROUND

This disclosure relates to the field of spinal fusion. In particular, this disclosure is drawn to spinal fusion devices and associated methods.

The spine is biomechanically a series of movable segments made up of vertebrae and discs. The collection of two adjacent vertebrae and their corresponding intervertebral disc is termed a spinal motion segment. Due to trauma, disease, or aging, the spine may require surgery. Conditions for which spinal fusion might be done include degenerative disc disease, treatment of a spinal tumor, a vertebral fracture, scoliosis, degeneration of the disc, spondylolisthesis, or any other condition that causes spinal instability. Restoration of spinal stability is often a goal of spinal surgery.

One procedure that is often used to surgically achieve spinal stability is a spinal fusion. Spinal fusion is a surgical technique used to permanently immobilize a spinal motion segment. Fusion occurs by eliminating the motion that naturally occurs through a spinal motion segment. A successful fusion requires both biological as well as mechanical alterations made to a spinal motion segment. Biologically, completion of the body's bone healing and remodeling process forms bone connecting the adjacent vertebrae of the motion segment. Supplemental bone tissue is typically used in conjunction with the patient's natural osteoblastic processes in a spinal fusion procedure. Mechanically, spinal instrumentation and spinal implants are commonly used to restore spinal alignment, disc height, and to achieve surgical stability of the operated spinal motion segment in order to allow for biological healing (spinal fusion). Once performed a spinal fusion can take up to two years for complete biological bone healing. During the time required for biological bone healing, the mechanical integrity conveyed by the spinal instrumentation and implants is paramount to maintain a favorable and stable environment to allow for successful biological bone healing.

The failure of the spinal fusion's healing process may cause a motion segment to remain mobile instead of intentionally immobilized. This failure of bone fusion is termed a pseudarthrosis and can cause persistent axial (back or neck) pain or neurological sequelae of pain, numbness, weakness, or loss of neurological function. Such clinical problems are termed symptomatic pseudarthrosis and may require additional treatment, incur additional costs for such treatment, and may require additional and risky revision spinal surgery. Scientific literature shows variability of the pseudarthrosis rate dependent on a multitude of clinical factors. However, it is reported to be as high as 30% in certain commonly performed spinal fusions.

Innovations that improve the likelihood of a successful spinal fusion can significantly improve clinical outcomes and reduce the cost of healthcare by reducing the need for additional costly and potentially risky treatments to treat pseudarthrosis. Such improvements can be made to spinal instrumentation and implants to promote a spinal fusion's healing or to improve the mechanical stability of the instrumentation or implant to the spine.

### SUMMARY

This disclosure relates to spinal fusion implants and related spinal fusion procedures for use in cervical, thoracic and lumbar applications. One type of spinal fusion is called an interbody fusion, or a fusion that is located in the area of the intervertebral disc that separates but connects two adjacent vertebrae. An interbody fusion procedure requires the intervertebral disc to be removed and the surfaces of the adjacent vertebrae (endplates) to be surgically prepared for bone healing. The space resulting from disc removal is called the intervertebral space. As part of the fusion operation a device is usually inserted into the intervertebral space, in the area of the removed intervertebral disc, to achieve spine alignment, spinal stability, and the desired space between the adjacent vertebrae. Such a device is called an interbody device or interbody fusion device, as it is positioned between the adjacent vertebrae. Additional material may be placed into the intervertebral space, often within the interbody device, to allow bone healing to connect the two adjacent vertebrae. The material may be osteoconductive (allows for a structural framework for biological bone healing), osteoinductive (biologically promotes bone healing), or both. Such material is generally described as bone graft or bone graft substitute. Fusion then occurs over time between the endplates of the vertebrae.

The interbody device itself may also be designed to mechanically or biologically integrate with the vertebral endplates in order to provide an additional point of healing or fixation across the intervertebral space. This is advantageous as it may improve the initial mechanical stability of the device to the bone and/or may provide a separate location for fusion to occur. By doing so it may prevent symptomatic pseudarthrosis if the bone graft or its substitute does not heal.

Generally, this disclosure describes an interbody fusion device that may be used for cervical, thoracic and lumbar interbody fusion. In one example, an interbody fusion device is a load bearing device shaped to fit between adjacent vertebrae in the location of the intervertebral disc. An opening is formed in the device between the upper and lower surfaces that can be filled with a material that will help to facilitate fusion of the vertebrae. In one example, the interbody fusion device is formed by two pieces as described below. Each piece, termed “First Piece” and “Second Piece” based on their place in an exemplary surgical insertion sequence, may be used together or independently of each other. Such flexibility is advantageous as it allows the surgeon to optimize the type of fixation best suited for the particular surgical scenario encountered when performing an interbody fusion.

The various embodiments of the interbody fusion devices describe herein may include one or more surfaces that have been treated with a surface treatment that promotes fusion between the interbody fusion device and the vertebrae. The surface treatment may be a spray-applied coating, such as, for example, a titanium plasma spray coating. Other treatments include laser etching. In general, the surface treatment increases porosity of the surface that is treated to promote bone ingrowth and/or bone ongrowth to the interbody fusion device. In some embodiments, one or more recessed pockets may be set into one or more surface of the interbody fusion device. To further promote fusion between the interbody fusion device and, for example, an endplate of a vertebra, a material may be applied to the treated surface(s) and/or the recessed pocket(s). The material may include bone material, bone marrow, bone substitutes (3-TCP, HA), bone morphonogenic protein (BMP), autologous stem cells, osteogenic growth factors, or other materials or combinations thereof.

In some embodiments, the second piece of the interbody fusion device, which is adapted to fit within the first piece of the interbody fusion device, may be utilized in an interbody fusion procedure without the first device. In such embodiments, the second piece may be adapted to receive removable bone anchors to help secure the second piece of the interbody fusion device between adjacent vertebrae.

### BRIEF DESCRIPTION OF THE DRAWINGS

FIG. 1 is an isometric view of one example of an interbody fusion device 10;

FIG. 2 is a side view of the interbody fusion device 10;

FIGS. 3A and 3B show exploded isometric views of the interbody fusion device 10;

FIGS. 4A and 4B are exploded top views of the interbody fusion device 10;

FIGS. 5A and 5B are exploded side views of the interbody fusion device 10;

FIG. 6A is an isometric view of one example of an interbody fusion device 100;

FIG. 6B is an exploded isometric view of the interbody fusion device 100;

FIG. 7A is an isometric view of one example of an interbody fusion device 200;

FIG. 7B is an exploded isometric view of the interbody fusion device 200;

FIG. 7C is a side view of the interbody fusion device 200;

FIG. 8A is an isometric view of one example of an interbody fusion device 300;

FIG. 8B is an isometric view of the interbody fusion device 300 that includes a porous material disposed within surface pockets;

FIG. 9A is an isometric view of one example of an interbody fusion device 400 from a first perspective;

FIG. 9B is an isometric view of the interbody fusion device 400 from a second perspective; and

FIG. 10 is an isometric view of one example of an interbody fusion device 500.

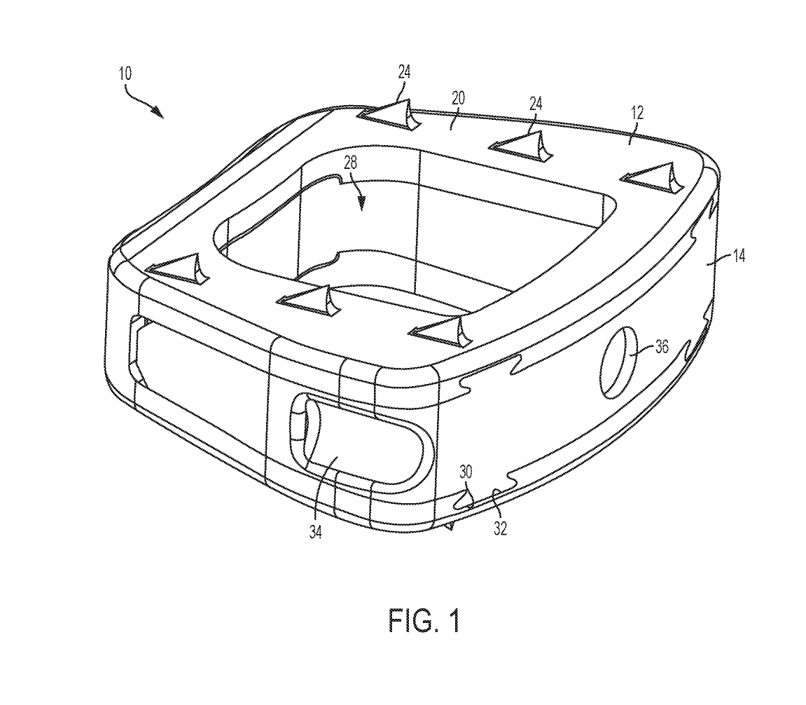

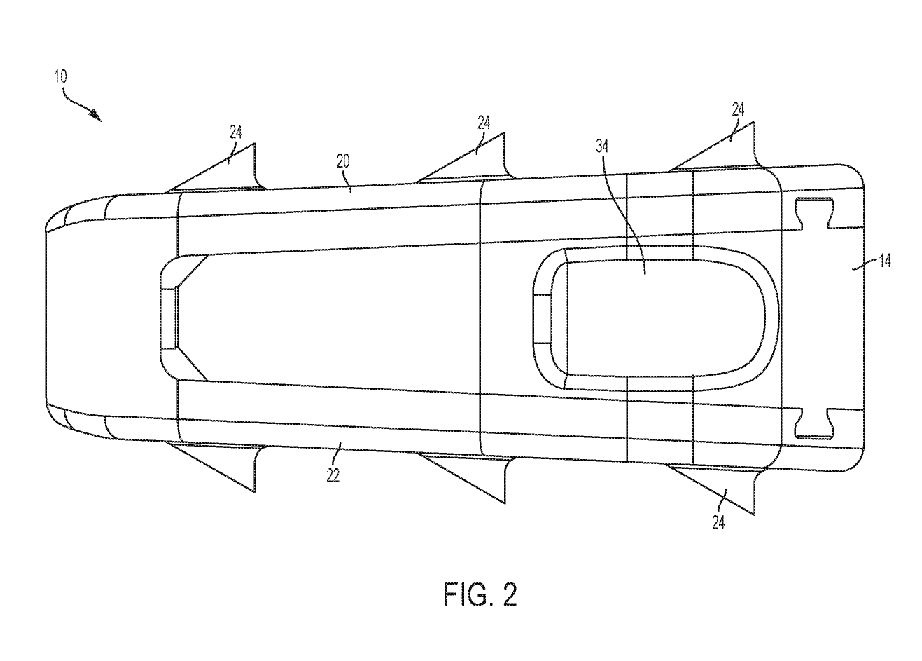

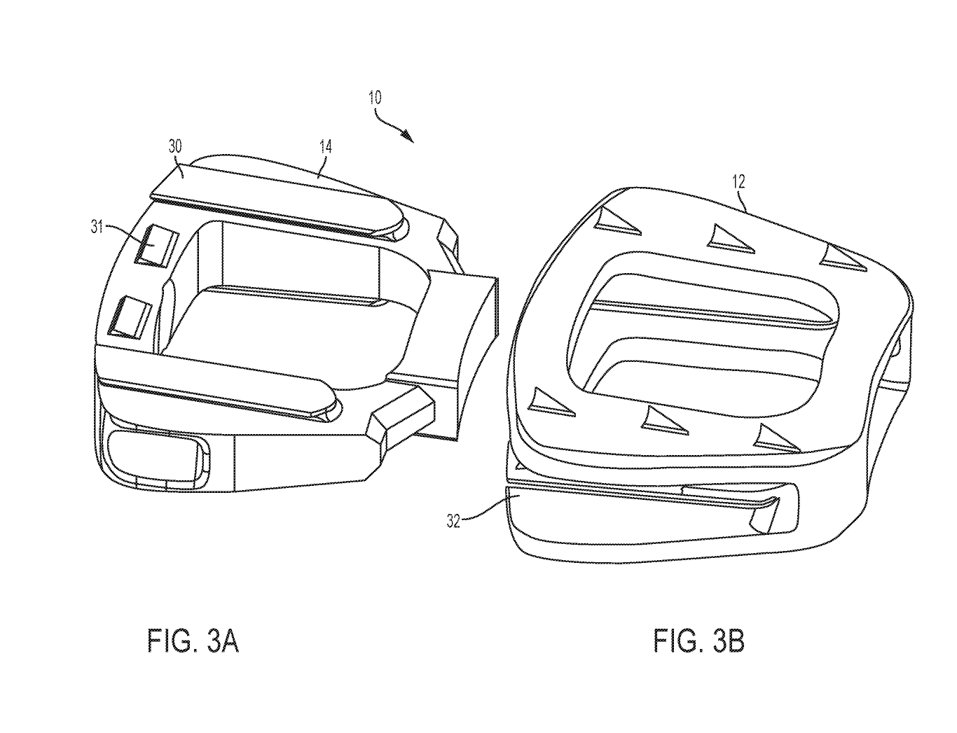

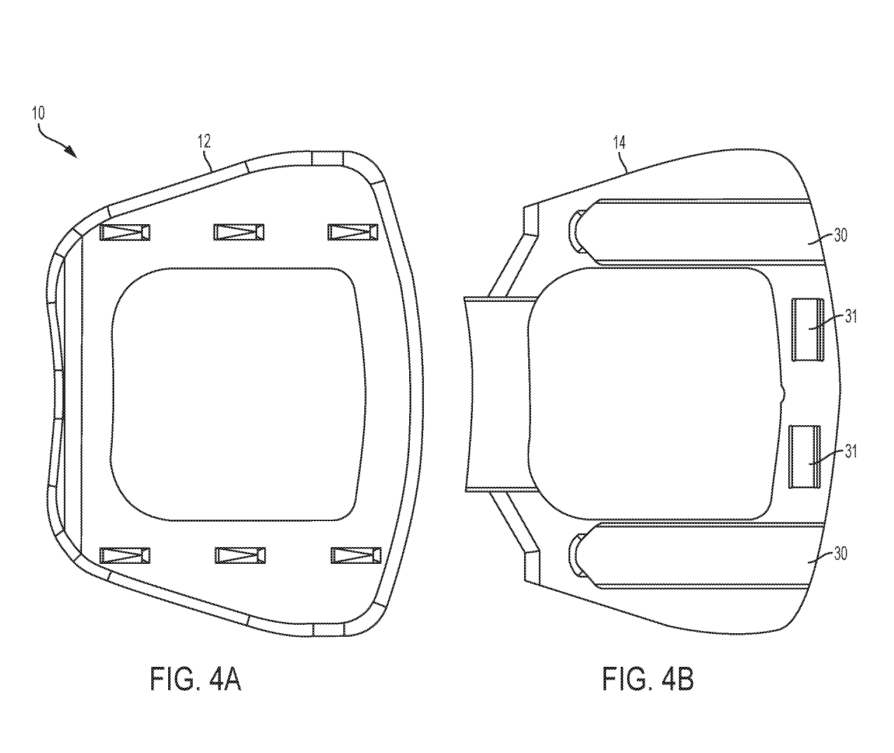

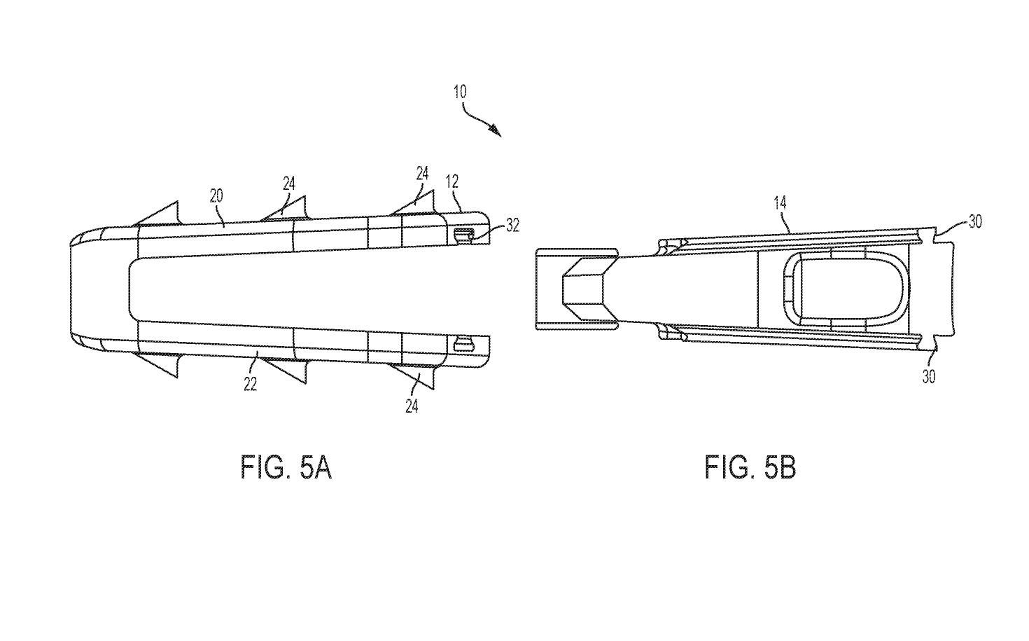

### DETAILED DESCRIPTION

The interbody fusion device 10 is designed to be inserted between the endplates of two adjacent vertebrae to provide the proper mechanical spacing and stability thereby promoting fusion between the vertebrae. The interbody fusion device 10, having the appropriate angle, size and shape, provides load bearing support as well as the proper spacing between the vertebrae. The interbody fusion device 10 is positioned between the end plates of the vertebrae within the vertebral body in the intervertebral space.

The interbody fusion device 10 can be formed by a first piece 12 and a second piece 14, inserted within the first piece 12. In one example, the first and second pieces 12 and 14 are comprised of different materials. In one example, the first piece 12 is made from titanium, which gives the interbody fusion device 10 strength. In one example, the second piece 14 is made from a biocompatible synthetic polymer, such as polyetheretherketone (PEEK®). If desired, the second piece 14 may include radio opaque 15 markers that will show up in an X-ray, although the first piece 12 (if made of titanium) will also show up in an X-ray. Note that the first and second pieces may also be comprised of other materials.

The first piece 12 has upper and lower surfaces 20 and 22, which are configured to engage the endplates of the vertebrae. A plurality of barbed teeth 24 extend from the upper and lower surfaces 20 and 22 to prevent anterior expulsion of the interbody fusion device 10. The barbed teeth 24 are configured to dig into the bone, resisting anterior forces, and provide immediate, mechanical post-operative resistance to anterior expulsion.

Although the second piece 14 may be designed to fit within the first piece 12, it may also be inserted and utilized independently of the first piece. In the examples shown, the first and second pieces 12 and 14 include sliding joints (similar to dovetail joints) to strengthen the union of the first and second pieces. The second piece 14 has two protrusions 30 shaped to fit within matching grooves 32 formed in the first piece 12. The second piece 14 also includes a plurality of angled protrusions 31 that lock into matching indentations formed in the first piece 12.

As shown in the figures, an opening 28 is formed in the interbody fusion device 10. The opening 28 provides a graft volume that can be filled with a prepared material that will help to facilitate fusion of the vertebrae through the opening 28. Examples of a material include bone material, bone marrow, bone substitutes ((3-TCP, HA), bone morphonogenic protein (BMP), autologous stem cells, osteogenic growth factors, or other materials or combinations thereof.

If desired, one or more openings can be formed in the interbody fusion device 10 to facilitate instrumentation devices. In the example shown in the figures, two lateral scallops 34 are formed on opposite sides of the second piece 14. A central scallop 36 is formed on the front surface of the second piece 14. The two lateral scallops 34 facilitate gripping the fusion device 10 using a bi-fed instrument grip (not shown), such as a Kerrison-style implant holder or a forceps style implant holder. The central scallop 36 facilitates manipulation of the fusion device 10 using an implant pusher (not shown). An implant pusher would typically have a dimple formed that matches the central scallop 36 to prevent slippage of the implant pusher.

As mentioned above, in one example, the first piece 12 can be comprised of titanium. To help the fixation of the fusion device 10 to the vertebrae, the upper and lower surfaces 20 and 22 of the first piece 12 can be at least partially textured or coated with a porous, biocompatible material or texturing that promotes bone in-growth to provide fixation to the bone. The titanium may also be coated with hydroxyapatite (HA) (or other material) to promote osseointegration. Likewise, the surface of the titanium can be in the form of porous titanium to permit bone ongrowth. The second piece 14 may also be at least partially textured or coated with a porous, biocompatible material or texturing that promotes bone in-growth to provide fixation to the bone.

The coating of the first piece or second piece 14 may be applied in a number of ways including but not limited to surface coating, surface modification such as an ingrained or etched surface, or as a separate piece that becomes physically attached to the primary piece (first or second).

The second piece may be used independently of and separately from the first piece such that it is not reliant on the presence of the first piece for efficacy or stability when inserted. It may be filled with material to promote a fusion. It may be used independently of the first piece regardless of whether it is textured or coated with material to promote bone to device fusion or mechanical stability. In and of itself, the second piece may be used independently as the only interbody device, coated or not, or it may be used in conjunction with the first piece. When used in conjunction with the first piece, the combination of the first and second piece may be inserted separately such that two distinct devices are inserted and then mated in-vivo or may be preassembled prior to insertion such that only one device is inserted.

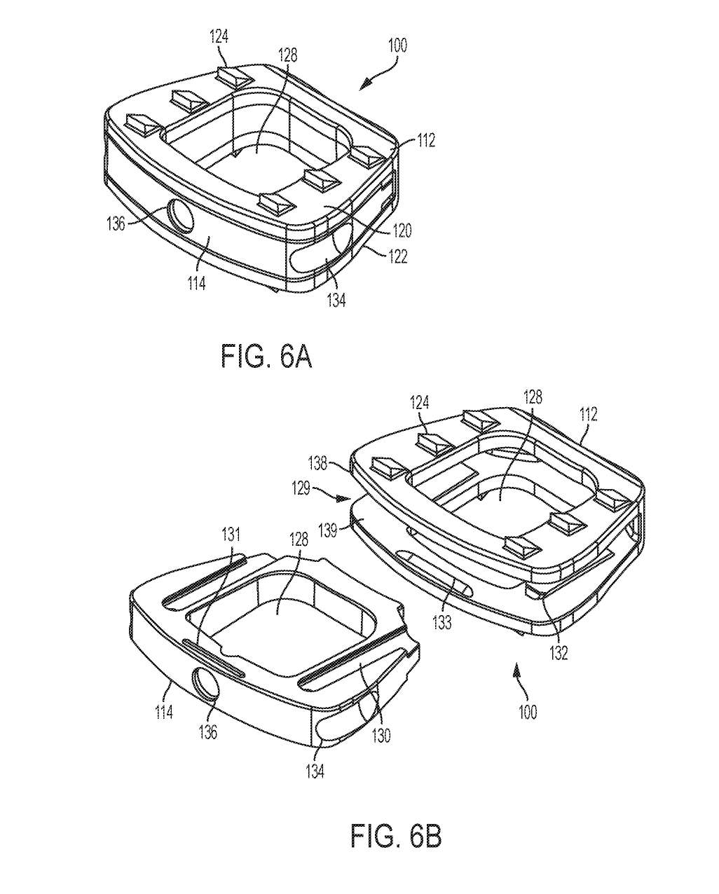

Referring now specifically to FIGS. 6A and 6B, isometric views of an example of an interbody fusion device 100 are shown. FIG. 6A shows the interbody fusion device 100 assembled. The interbody fusion device 100 comprises an interbody housing or first piece 112 and a body or second piece 114. When assembled, the second piece 114 is inserted within an inner void 129 of the first piece 112. The inner void 129 includes an upper inner surface 138 and a lower inner surface 139. In some embodiments, the first and second pieces 112 and 114 are comprised of different materials. In some embodiments, the first piece 112 may be made from, for example, titanium, which gives the interbody fusion device 100 strength. To help the fixation of the fusion device 100 to the vertebrae, the upper and lower surfaces 120 and 122 of the first piece 112 can be at least partially textured or coated with a porous, biocompatible material or texturing that promotes bone in-growth to provide fixation to the bone. The titanium may also be coated with hydroxyapatite (HA), or other material, to promote osseointegration. Likewise, the surface of the titanium can be in the form of porous titanium to permit bone ongrowth.

The second piece 114 may also be at least partially textured or coated with a porous, biocompatible material or texturing that promotes bone in-growth to provide fixation to the bone. The coating of the first piece 112 or second piece 114 may be applied in a number of ways including but not limited to surface coating, surface modification, such as an ingrained or etched surface, or as a separate piece that becomes physically attached to the primary piece (e.g., the first piece 112 or the second piece 114). In some embodiments, the second piece 114 may be made from, for example, a biocompatible synthetic polymer, such as PEEK®. In some embodiments, the second piece 114 may include radio opaque markers that will show up in an X-ray, although the first piece 112 (if made of titanium) will also show up in an X-ray. Note that the first and second pieces 112 and 114 may also be comprised of other materials.

In some embodiments, the first piece 112 has upper and lower surfaces 120 and 122, respectively, which are configured to engage endplates of adjacent vertebrae. In some embodiments, a plurality of barbed teeth 124 extend from the upper and lower surfaces 120 and 122, respectively, to prevent anterior expulsion of the interbody fusion device 100. The barbed teeth 124 are configured to dig into the bone, resisting anterior forces, and provide immediate, mechanical post-operative resistance to anterior expulsion.

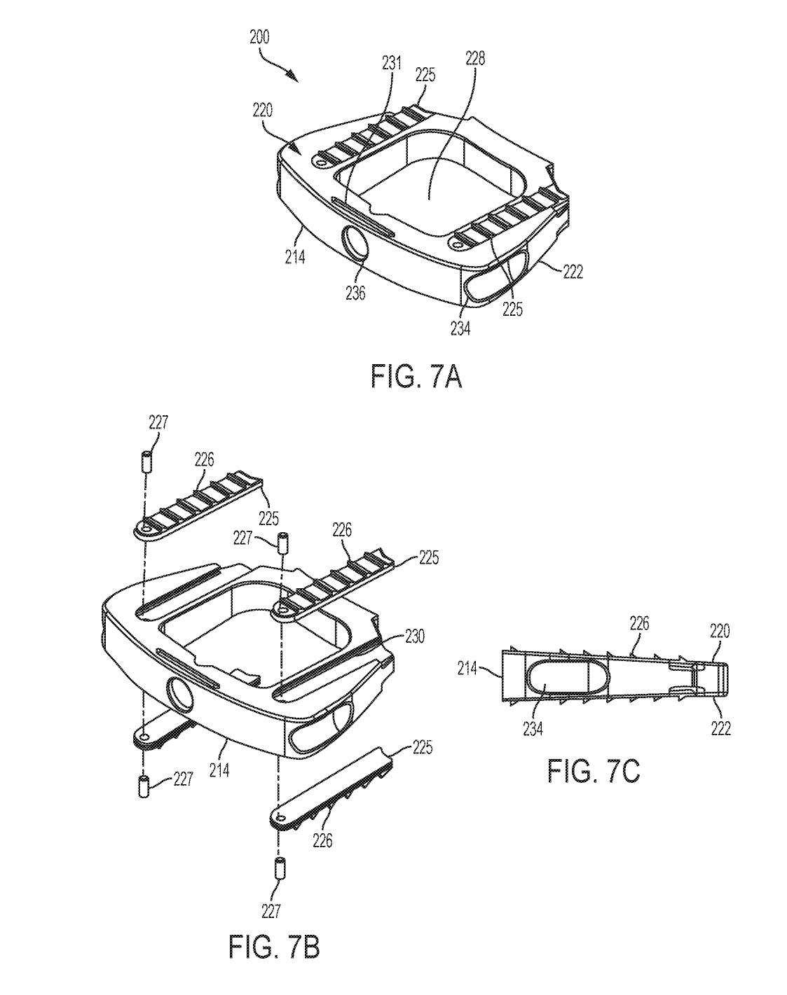

Although the second piece 114 is designed to fit within the first piece 112, it may also be inserted into a surgical site and utilized independently of the first piece 112 (e.g., see the embodiment of FIGS. 7A-7C). In some embodiments, the second piece 114 includes one or more depressions formed into the upper and lower surfaces 120 and 122. As shown in FIGS. 6A and 6B, the depressions are grooves 130. The grooves 130 are adapted to receive matching protrusions 132 of the first piece 112 to secure the second piece 114 to the first piece 112. In some embodiments, the second piece includes two grooves 130 formed into the upper surface 120 and two grooves 130 formed into the lower surface 122. In such embodiments, the first piece 112 includes corresponding matching protrusions 132. The grooves 130 can comprise various shapes that ensure an interlocking engagement with the matching protrusions 132, such as, for example, dovetail, “T”, and the like.

The second piece 114 also includes a ridge 131 that is adapted to engage a depression 133 in the first piece 112. Engagement of the ridge 131 with the depression 133 helps secure the second piece 114 to the first piece 112. In some embodiments the first piece 112 includes more than one depression 133. For example, multiple depressions 133 can be disposed on a first surface of the first piece 112, or opposing depressions 133 can be disposed on opposing surfaces of the first piece 112. In either case, corresponding ridges 131 are included on the second piece 114.

As shown in the FIGS. 6A and 6B, an opening 128 is formed through the first piece 112 and the second piece 114. The opening 128 provides a graft volume that can be filled with a prepared material that will help to facilitate fusion of the vertebrae through the opening 128. Examples of a prepared material include bone material, bone marrow, bone substitutes (3-TCP, HA), bone morphonogenic protein (BMP), autologous stem cells, osteogenic growth factors, or other materials or combinations thereof.

In some embodiments, one or more openings and/or features can be formed in the interbody fusion device 100 to facilitate handling of the interbody fusion device 100 with instrumentation devices. As shown in FIGS. 6A and 6B, two lateral scallops 134 are formed on opposite sides of the second piece 114. A central opening or scallop 136 is formed on a front of the second piece 114. The two lateral scallops 134 facilitate gripping of the fusion device 100 with a bi-fed instrument grip (not shown), such as a Kerrison-style implant holder or a forceps style implant holder. The central scallop 136 facilitates manipulation of the fusion device 100 using an implant pusher (not shown). An implant pusher would typically have a dimple formed that matches the central scallop 136 to prevent slippage of the implant pusher.

Referring now generally to FIGS. 7A-7B, isometric views of one example of an interbody fusion device 200 are shown. The interbody fusion device 200 comprises a body 214 that is similar to the second piece 114 of FIGS. 6A and 6B and comprises similar features. For example, the body 214 includes upper and lower surfaces 220 and 222, respectively, that are adapted to engage endplates of adjacent vertebrae. The interbody fusion device 200 further includes an opening 228 formed through the interbody fusion device 200 that is similar to the opening 128.

In some embodiments, the interbody fusion device 200 includes one or more protrusions 231 that are adapted to dig into the adjacent vertebrae to help secure the interbody fusion device 200 in place. In some embodiments, the interbody fusion device 200 further includes a pair of removable bone anchors 225 that can be inserted into depressions formed into the upper and lower surfaces 220 and 222. In some embodiments, the depressions are grooves 230 that are similar to the grooves 130 discussed relative to FIGS. 6A and 6B. In some embodiments, the removable bone anchors 225 include one or more protrusions 226 that are adapted to dig into endplates of the adjacent vertebrae, similar to the barbed teeth 124. In some embodiments, each of the removable bone anchors 225 may be secured to the body 214 via a pin 227. The pins 227 are adapted to prevent the removable bone anchors 225 from sliding out of the body 214. The pins 227 may be press fit through the removable anchors 225 and the body 214. In some embodiments, the body 214 may include a pair of lateral scallops 234 and/or a central opening or scallop 236. The pair of lateral scallops 234 and the scallop 236 are similar to the pair of lateral scallops 134 and the scallop 136.

Referring now to FIG. 7C, a side view of the interbody fusion device 200 is shown. In some embodiments, the upper and lower faces 220 and 222 of the body 214 are parallel to one another. In some embodiments, the upper and lower faces 220 and 222 are angled relative to one another as shown in FIG. 7C. The angle between the upper and lower faces 220 and 222 may be varied depending upon various design considerations. For example, the angle between the upper and lower faces 220 and 222 may be varied to help achieve a desired angle between the endplates of the adjacent vertebrae.

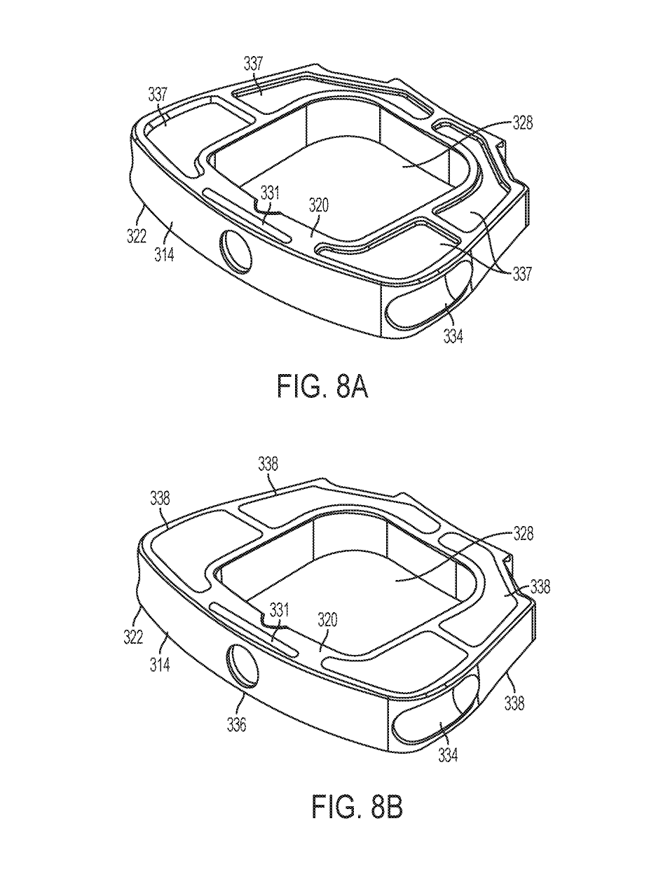

Referring now to FIGS. 8A and 8B, isometric views of one example of an interbody fusion device 300 are shown. The interbody fusion device 300 comprises a body 314 that includes upper and lower surfaces 320 and 322, respectively, that are adapted to engage endplates of adjacent vertebrae. The interbody fusion device 300 further includes an opening 328 formed through the interbody fusion device 300 that is similar to the opening 128. In some embodiments, the body 314 may include a pair of lateral scallops 334 and/or a central opening or scallop 336. The pair of lateral scallops 334 and the scallop 336 are similar to the pair of lateral scallops 134 and the scallop 136. In some embodiments, a ridge 331 may be included on either or both of the upper and lower surfaces 320 and 322. The ridge 331 interacts with the endplates of the adjacent vertebrae to help secure the interbody fusion device 300 in place.

The interbody fusion device 300 may also include one or more depressions formed into the upper and lower surfaces 320 and 322. In some embodiments, the one or more depressions may be one or more recessed pockets 337. The one or more recessed pockets 337 act as cavities in which a material 338 may be inserted. FIG. 8B shows the material 338 inserted into the one or more recessed pockets 337.

In some embodiments, the one or more recessed pockets 337 may include a surface treatment that promotes direct fusion of the interbody fusion device 300 with the endplates of the adjacent vertebrae. This direct fusion may be complementarily to any osteoconductive material that is placed within the opening 328. The material 338 may be topically applied or actually mechanically incorporated onto a surface of the one or more recessed pockets 337. The one or more recessed pockets 337 can house a porous type material or may include a porous etching into the surface of the one or more recessed pockets 337. In some embodiments, a titanium-plasma-spray coating may be applied to the one or more recessed pockets 337. In some embodiments, a process to mechanically fix a surface material onto the one or more pockets 337 and/or the body 314 may include a mechanical (e.g., laser etching) or biological modifications or enhancements that enable bone ingrowth into the interbody fusion device 300. In some embodiments, the interbody fusion device 300 may undergo post processing to remove excess material, thus leaving only the porous material within the one or more recessed pocket 337. Treating surfaces of the body 314 is advantageous because it reduces a risk of the treatment partially or completely sheering off when inserted into an interbody space.

In some embodiments, the material 338 promotes fusion between the interbody fusion device 300 and endplates of adjacent vertebrae. The material 338 may include one or more of the following: osteoinductive materials, osteoconductive materials, bone grafts, bone graft substitutes, biological proteins and substrates, and other mechanical or biological modifications or enhancements that enable bone ingrowth into the interbody fusion device 300. In some embodiments, the material 338 supplements fusion between the endplates that occurs from inclusion of a bone graft material within the opening 338.

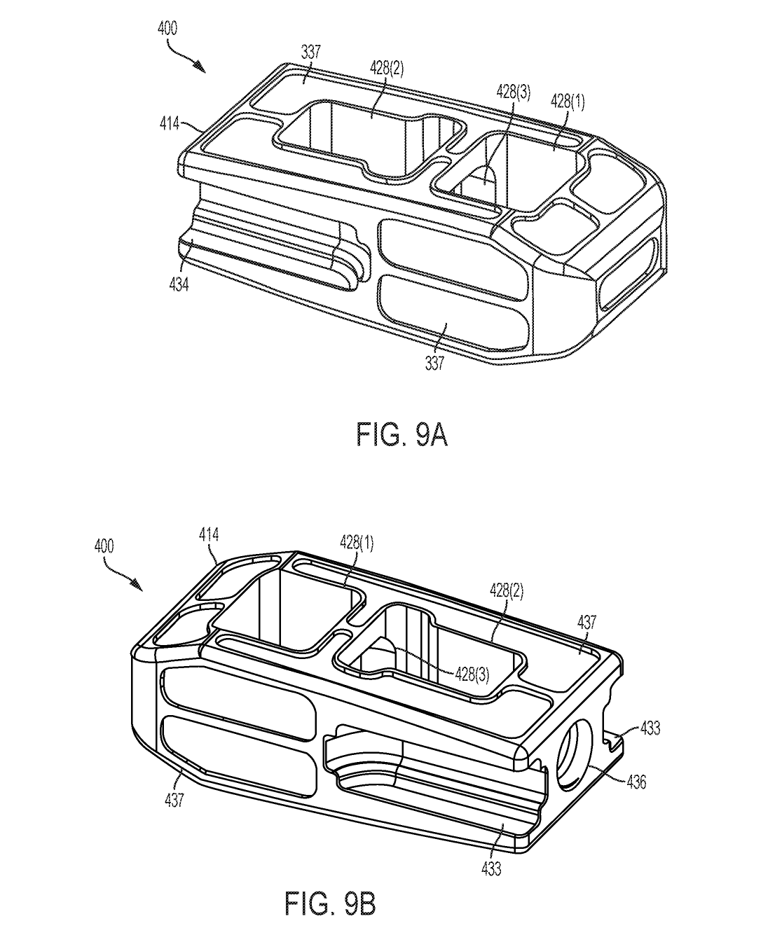

FIGS. 9A and 9B are isometric views of one example of an interbody fusion device 400 that may be used for posterior lumbar interbody fusion or transforaminal lumbar interbody fusion. The interbody fusion device 400 comprises a body 414. In some embodiments, the body 414 may include a pair of lateral scallops 434 and/or a central opening or scallop 436. The pair of lateral scallops 434 and the scallop 436 are similar to the pair of lateral scallops 134 and the scallop 136.

In some embodiments, the body 414 includes an opening 428(1) formed through the body 414 to permit placement of a graft volume that can be filled with a prepared material that will help to facilitate fusion of the vertebrae through the opening 428(1). In some embodiments, an opening 428(2) may also be included. In some embodiments, the opening 428(2) may be interconnected with the opening 428(1) by an opening 428(3). In some embodiments, the openings 428(1)-(3) may be replaced with a single, larger opening.

The interbody fusion device 400 may also include one or more depressions formed into surfaces of the body 414. In some embodiments, the one or more depressions may be recessed pockets 437 that are substantially similar to the one or more recessed pockets 337. The one or more recessed pockets 437 may be included on any of the surfaces of the body 414. For example, as shown in FIGS. 7A and 7B, the one or more recessed pockets 337 may be placed on a top, side, or bottom of the body 414. A material, similar to the material 338, may be introduced into the one or more recessed pockets 437 to promote direct fusion of the interbody fusion device 400 with endplates of adjacent vertebrae and/or with a spinal disc.

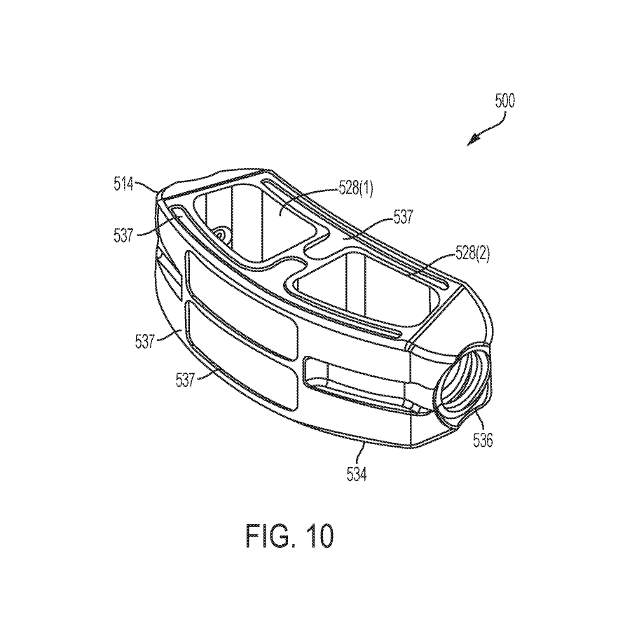

FIG. 10 is an isometric view of one example of an interbody fusion device 500 that may be used for transforaminal lumbar interbody fusion. The interbody fusion device 500 comprises a body 514. In some embodiments, the body 514 may include a pair of lateral scallops 534 and/or a central opening or scallop 536. The pair of lateral scallops 534 and the scallop 536 are similar to the pair of lateral scallops 134 and the scallop 136.

In some embodiments, the body 514 includes an opening 528(1) formed through the body 514 to permit placement of a graft volume that can be filled with a prepared material that will help to facilitate fusion of the vertebrae through the opening 528(1). In some embodiments, an opening 528(2) may also be included. In some embodiments, the opening 528(2) may be interconnected with the opening 528(1) by an opening similar to the opening 428(3). In some embodiments, the openings 528(1)-(2) may be replaced with a single, larger opening.

The interbody fusion device 500 may also include one or more depressions formed into surfaces of the body 514. In some embodiments, the one or more depressions may be recessed pockets 537 that are substantially similar to the one or more recessed pockets 337. The one or more recessed pockets 537 may be included on any of the surfaces of the body 514. For example, as shown in FIG. 10, the one or more recessed pockets 537 may be placed on a top, side, or bottom of the body 514. A material, similar to the material 338, may be introduced into the one or more recessed pockets 537 to promote direct fusion of the interbody fusion device 500 with endplates of adjacent vertebrae and/or with a spinal disc.

As will be appreciated by those having skill in the related arts, the various interbody fusion devices described above can be treated with a porous type material or may include a porous etching into the surface of the interbody fusion device. In some embodiments, a titanium-plasma-spray coating may be applied to the one or more surfaces of the interbody fusion devices. Such treatments may be applied to one or more of the first piece and the second piece of the interbody fusion device. In some embodiments, the first piece may include a recessed pocket, such as, for example, a recessed pocket similar to the recessed pocket 337.

The following is a description illustrating how an interbody fusion device may be used in a spinal fusion procedure. First, the vertebral body is prepared for the implant. This preparation may include cleaning out the disc, nucleus pulposus and preparing the endplates of the vertebral bodies. If desired, a prepared material can be placed in the opening 28 of the fusion device 10 to promote fusion. The implant is inserted between the adjacent vertebrae using the appropriate instrumentation, as desired.

The fusion biologically occurs between the vertebral endplates via bone healing from each endplate that connects through the “material promoting fusion”, including but not limited to bone graft, bone graft substitutes, and biological proteins and substrates intended to promote bone growth, which is placed inside of the interbody device. Pseudarthrosis, or a failure of bone healing through the interbody device, can occur and often leads to poor surgical outcomes with persistent pain and neurological deficits. The device described is clinically advantageous and different from the available technology in that it promotes structural integrity via healing between the device itself and the vertebral endplate. This is in addition to the expected traditional healing process that occurs through the “material promoting fusion” that is placed within the interbody device and causes biological bone healing between the vertebral endplates. By providing a separate location for healing between the interbody device itself and the vertebral endplate, this device supplements the usual healing pathway in order to minimize the clinical detriments of pseudarthrosis.

In the preceding description, the devices and methods are described with reference to exemplary embodiments thereof. Various modifications and changes may be made thereto without departing from the broader spirit and scope of the disclosure.

What is claimed is:

1. An interbody fusion device for fusion of vertebrae, comprising:
a body comprising an upper surface and a lower surface, wherein the upper and lower surfaces are adapted to contact endplates of two opposing vertebrae;
an opening formed through the body that breaks a plane of the upper and lower surfaces; and
a depression formed into at least one of the upper and lower surfaces of the body, wherein the depression is adapted to receive a component to secure the component to the body.

2. The interbody fusion device of claim 1, wherein the depression is a groove.

3. The interbody fusion device of claim 2, wherein the component is a removable bone anchor that is adapted to engage the groove.

4. The interbody fusion device of claim 3, wherein the removable bone anchor comprises barbed teeth adapted to engage an endplate of a vertebrae.

5. The interbody fusion device of claim 1, wherein the component is a housing comprising:
an inner void formed between an upper inner surface of the housing and a lower inner surface of the housing, wherein the inner void is adapted to receive the body; and
a protrusion attached to either of the upper and lower inner surfaces of the housing, wherein the at least one protrusion is adapted to engage the depression to secure the housing to the body.

6. The interbody fusion device of claim 5, wherein the housing comprises barbed teeth affixed to an upper outer surface and a lower outer surface

7. The interbody fusion device of claim 5, further comprising:
a ridge disposed on either of the upper and lower surfaces of the body; and
a depression adapted to receive the ridge when the body is inserted into the housing.

8. The interbody fusion device of claim 1, further comprising a pair of scallops adapted for interaction with an instrument.

9. The interbody fusion device of claim 1, wherein the depression is a recessed pocket.

10. The interbody fusion device of claim 9, wherein the device comprises at least one recessed pocket disposed on the upper surface of the body and at least one recessed pocket disposed on the lower surface of the body.

11. The interbody fusion device of claim 9, further comprising a recessed pocket disposed on a side of the body.

12. The interbody fusion device of claim 1, wherein at least one of the upper surface and the lower surface comprise of porous surface treatment adapted to promote fusion between the interbody fusion device and at least one of the two opposing vertebrae.

13. The interbody fusion device of claim 12, wherein the porous surface treatment is a titanium plasma spray coating.

14. The interbody fusion device of claim 9, wherein the recessed pocket includes a porous surface.

15. The interbody fusion device of claim 14, wherein the porous surface treatment is a titanium plasma spray coating.

16. The interbody fusion device of claim 9, further comprising a material disposed within the recessed pocket, wherein the material is adapted to promote fusion between the device and an endplate of the two opposing vertebrae.

17. The interbody fusion device of claim 16, wherein the material may be selected from the group consisting of: osteoinductive materials, osteoconductive materials, bone grafts, bone graft substitutes, and biological proteins and substrates.

18. A method of fusing vertebrae, comprising:
removing at least a portion of a disc between first and second vertebrae, providing an interbody fusion device comprising:
a body comprising an upper surface and a lower surface, wherein the upper and lower surfaces are adapted to contact endplates of two opposing vertebrae;
an opening formed through the body that breaks a plane of the upper and lower surfaces; and
a depression formed into at least one of the upper and lower surfaces of the body, wherein the depression is adapted to receive a component to secure the component to the body;
inserting the first piece between the vertebrae,
packing the second piece with material to facilitate fusion of the first and second vertebrae, and
inserting the second piece fully into the first piece to mate the first and second pieces.

19. The method of claim 18, wherein the component is a housing comprising:
an inner void formed between an upper inner surface of the housing and a lower inner surface of the housing, wherein the inner void is adapted to receive the body; and
a protrusion attached to either of the upper and lower inner surfaces of the housing, wherein the at least one protrusion is adapted to engage the depression to secure the housing to the body.

20. The method of claim 18, wherein interbody fusion device comprises a porous surface treatment adapted to promote fusion between the interbody fusion device and at least one of the two opposing vertebrae.
